(19) United States
(12) Patent Application Publication
(10) Pub. No.: US 2024/0269412 A1
(43) Pub. Date: Aug. 15, 2024

(54) Intubation Stylet and Related Methods

(71) Applicants: Patrick John Keating, Rockport, MA (US); Benjamin C. Levie, Gloucester, MA (US)

(72) Inventors: Patrick John Keating, Rockport, MA (US); Benjamin C. Levie, Gloucester, MA (US)

(21) Appl. No.: 18/108,817

(22) Filed: Feb. 13, 2023

(51) Int. Cl. A61M 16/04

(52) U.S. Cl. CPC A61M 16/0488

(57) ABSTRACT

The invention is an improved stylet with a firm and easily moldable portion attached to a tip constructed of a shape-memory alloy, where the tip is designed to deflect inside a patient's body to ease the insertion of an endotracheal tube with minimal trauma to the patient. The invention also includes a method of intubating a patient to minimize trauma.

### BACKGROUND

### 1. Field

The present disclosure relates to the intubation of a patient, specifically to an improved intubation stylet and related methods.

### 2. Discussion of Prior Art

Medical practitioners presently insert endotracheal tubes in patients by placing a stylet inside an endotracheal tube and then manipulating the stylet to a desired shape. Stylets are generally manipulated into a fixed shape and maneuvered to the vocal cords and allowing the endotracheal tube to pass over the stylet, where the tube is used to ventilate the lungs through the trachea. The existing stylets are rigid during insertion, making the accurate insertion of an endotracheal tube difficult in many patients. As such, prior art stylets are undesirable because of their fixed shape, which can cause abrasions in placing the stylet at the glottic opening.

The present invention is directed at overcoming, or at least improving upon, the disadvantages of the prior art.

### BRIEF SUMMARY

The present invention accomplishes this goal by providing an improved stylet comprising a proximate portion 11 configured to be firm and easily moldable and a distal portion 12 comprising one of a: thermal activating alloy and a shape-memory alloy; the distal portion having a thermal mass; and wherein the distal portion if configured to deflect at a predetermined temperature based in part on its thermal mass. Preferably, the distal portion 12 can be constructed using nitinol.

The disclosure includes an improved intubation stylet, which comprises a proximate portion configured to be firm and easily moldable; a distal portion fixed to the proximate portion comprising portion comprising a shape-memory alloy; the distal portion having a thermal mass; the distal portion having curvature in a first direction and a shape memory in a second direction, where the second direction is opposite the first direction; wherein at least a portion of the distal portion is configured to deflect from the first direction to the second direction at a predetermined temperature based in part on its thermal mass; a tube slidably attached over the surface of the intubation stylet; and wherein the intubation stylet is configured to also deflect the tube from the first direction to the second direction at the predetermined temperature. In some embodiments, the distal portion can be configured to be firm and easily moldable prior to its deflection at the predetermined temperature. In some embodiments, the first direction is an anterior direction. In some embodiments, the second direction is a posterior direction. The intubation stylet can further comprise an external heat source fixed to an exterior surface of the intubation stylet and configured to heat the intubation stylet to the predetermined temperature.

This disclosure also includes a method of intubating a patient, the steps including: providing an intubation stylet comprising: a proximate portion configured to be easily moldable by hand; a distal portion fixed to the proximate portion comprising a shape-memory alloy; the distal portion having a thermal mass; the distal portion having curvature in a first direction and a shape memory in a second direction, where the second direction is opposite the first direction; and wherein at least a portion of the distal portion is configured to deflect from the first direction to the second direction at a predetermined temperature based in part on its thermal mass; a tube slidably attached over the surface of the intubation stylet; wherein the intubation stylet is configured to also deflect the tube from the first direction to the second direction at the predetermined temperature; manipulating the proximate portion so that the distal portion points in an anterior direction; deflecting the distal portion to a position that facilitates positioning the distal portion at a glottic opening of the patient by heating the distal portion to the predetermined temperature; and sliding the tube over the intubation stylet and through the patient's vocal cords. In some embodiments, the distal portion is configured to be firm and easily moldable prior to its deflection at the predetermined temperature. In some embodiments, the first direction is an anterior direction. In some embodiments, the second direction is a posterior direction. In some embodiments, the intubation stylet further comprises an external heat source fixed to an exterior surface of the intubation stylet and configured to heat the intubation stylet to the predetermined temperature.

The disclosed improved intubation stylet 10 and related methods, in some respects, has the benefit of a deflection angle that provides superior positioning, allowing the tube to pass through the glottic opening instead of towards the Anterior Commissure. The Anterior Commissure is the point at which the true vocal folds insert into the thyroid cartilage in the midline, approximately halfway between the most inferior aspect of the thyroid notch and the inferior border of the thyroid cartilage.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is an exemplary side view of the improved intubation stylet prior to the distal portion deflecting.

FIG. 2 is an exemplary side view of the improved intubation stylet after the distal portion has been deflected.

FIG. 3 is an exemplary side view of the improved intubation stylet prior to the distal portion deflecting and with an exemplary breathing tube positioned over the stylet.

FIG. 4 is an exemplary side view of the improved intubation stylet after the distal portion has been deflected and with an exemplary breathing tube positioned over the stylet, showing that the improved intubation stylet also deflects a tube placed over it.

FIG. 5 is a chart depicting a method of using the disclosed improved intubation stylet.

### DETAILED DESCRIPTION

Illustrative embodiments are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. The improved intubation stylet disclosed herein boasts a variety of inventive features and components that warrant patent protection, both individually and in combination.

The present disclosure relates to an improved intubation stylet with a malleable portion and a wire tip configured to change shape in reaction to the patient's body temperature to ease insertion.

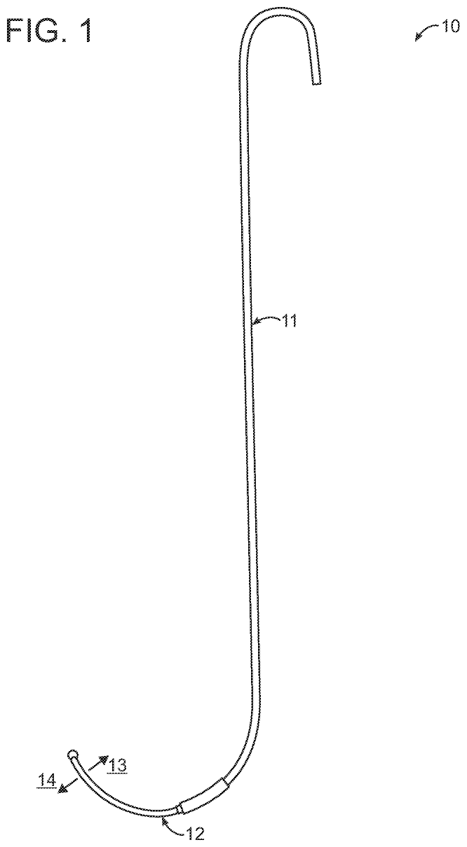

In FIG. 1 is an exemplary side view of the improved intubation stylet 10 which comprises a proximate portion 11 configured to be firm and easily moldable and a distal portion 12 fixed to the proximate portion 11 comprising a shape-memory alloy; the distal portion having a thermal mass, where the distal portion 12 has a curvature in a first direction and a shape memory in a second direction, where the second direction is opposite the first direction. At least a portion of the distal portion can be configured to deflect from the first direction to the second direction at a predetermined temperature based in part on its thermal mass. In some embodiments, the predetermined temperature can also be based, in part, on: a normal body temperature for a human being within a reasonable range, such as 98.6° F.+/−10° F. and/or a room temperature, such as a predetermined temperature of 64° F. or above to account for a normal operating room temperature of 64° F. In some embodiments, the distal portion 12 can further comprise nitinol. The distal portion can be configured to be firm and easily moldable prior to its deflection at the predetermined temperature. The distal portion 12 can be configured as a curved portion that at least partially reverses its direction of curvature. For example, the distal portion 12 can be manufactured with a curvature in a first direction 13 and configured to deflect in a second direction 14 when it reaches the predetermined temperature. The distal portion 12 can be configured as a straight portion that deflects into a curved portion. The distal portion 12 can be configured as a straight portion that deflects in a posterior direction. In some embodiments, an exterior heater can be attached to the improved intubation stylet 10 to speed its temperature change to the predetermined temperature. In some embodiments the improved intubation stylet 10 can be heated passively by the patient's body heat.

The proximate portion 11 and distal portion 12 can be firm and easily moldable to ease insertion into the body. Once deflected, the distal portion 12 can become firm and difficult to mold by hand.

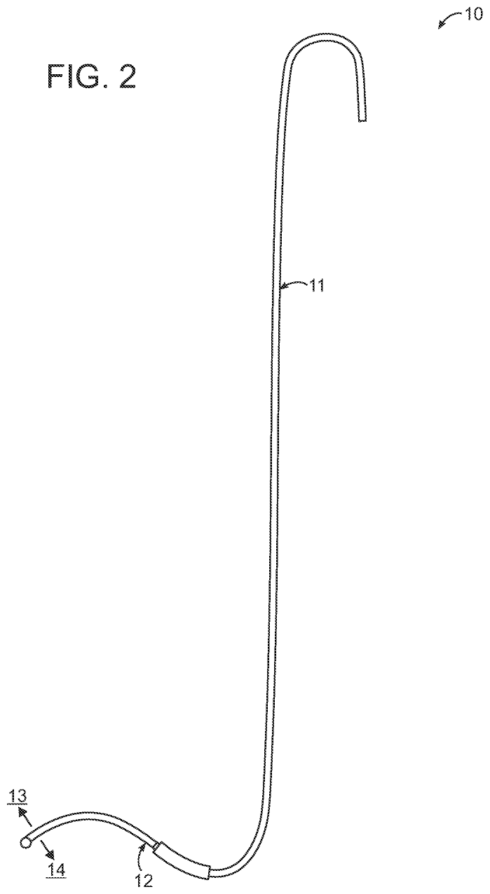

In FIG. 2 is an exemplary improved intubation stylet 10 where the distal portion 12 has been deflected at the predetermined temperature. In this example, the distal portion 12 at least partially deflects in the second direction 14. Depending on the application, the first direction 13 can represent an anterior direction in a patient and the second direction 14 can represent a posterior direction in a patient.

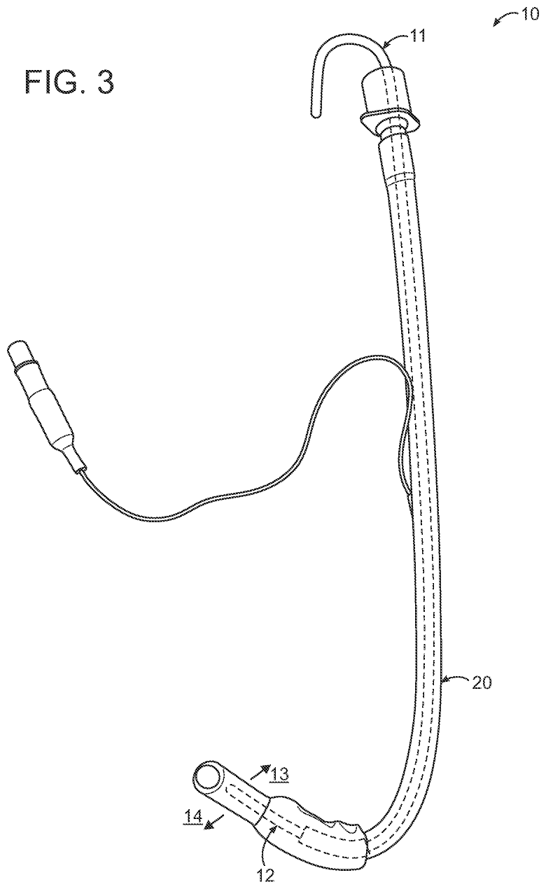

In FIG. 3 is an exemplary improved intubation stylet 10 sheathed in an exemplary breathing tube 20. It is contemplated that one use of this improved intubation stylet 10 is for it to be sheathed in a breathing tube 20, allowing the breathing tube 20 to be brought to a patient's glottic opening in a first attempt with minimal trauma to a patient's throat and vocal cords.

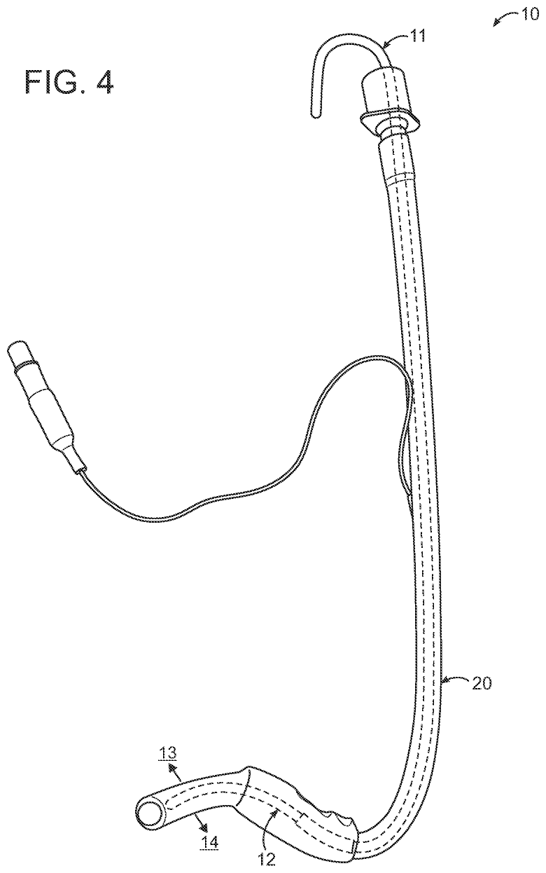

FIG. 4 is an exemplary improved intubation stylet 10 sheathed in an exemplary breathing tube 20 after the distal portion 12 has been deflected. The improved intubation stylet 10 can be configured to also deflect the breathing tube 20 to position the breathing tube 20 for insertion through a patient's vocal cords, while the distal portion 12 of the improved intubation stylet 10 remains at the glottic opening. It is contemplated that the improved intubation stylet 10 and breathing tube 20 would be inserted into a patient while in a shape similar to FIG. 3 and passively or actively heated to the predetermined temperature in situ. FIG. 4 represents a potential shape of the improved intubation stylet sheathed in a breathing tube 20 after the distal portion 12 deflects at the predetermined temperature. In FIG. 4, the distal portion 12 can easily be maneuvered to the patient's glottic opening and the breathing tube 20 slid through glottic opening.

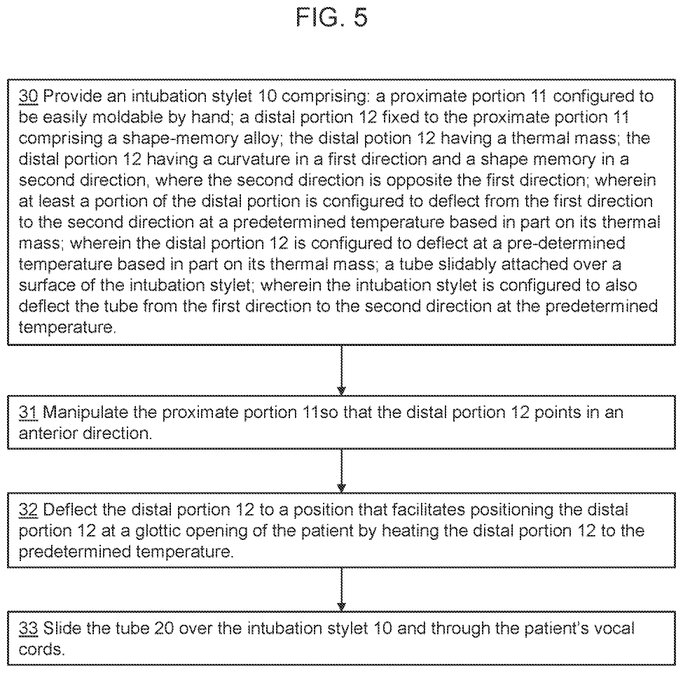

In FIG. 5 is an improved method of intubating a patient to minimize intubation attempts and trauma to the patient. The steps of the method include the following without regard to order:
In step 1 30: Provide an intubation stylet 10 comprising: a proximate portion 11 configured to be easily moldable by hand; a distal portion 12 fixed to the proximate portion 11 comprising a shape-memory alloy; the distal portion 12 having a thermal mass; the distal portion 12 having a curvature in a first direction and a shape memory in a second direction, where the second direction is opposite the first direction; wherein at least a portion of the distal portion is configured to deflect from the first direction to the second direction at a predetermined temperature based in part on its thermal mass; wherein the distal portion 12 is configured to deflect at a pre-determined temperature based in part on its thermal mass; a tube slidably attached over a surface of the intubation stylet; wherein the intubation stylet is configured to also deflect the tube from the first direction to the second direction at the predetermined temperature.
In step 2 31: Manipulate the proximate portion 11 so that the distal portion 12 points in an anterior direction.
In step 3 32: Deflect the distal portion 12 to a position that facilitates positioning the distal portion 12 at a glottic opening of the patient by heating the distal portion 12 to the predetermined temperature.
In step 4 33: Slide the tube 20 over the intubation stylet 10 and through the patient's vocal cords.

This method can be used to accurately insert breathing tubes into patients by using the improved intubation stylet's 10 curvature in an anterior direction, whether manufactured or manipulated, to reduce the chance that the breathing tube is accidentally inserted into the esophagus. The shape of the improved intubation stylet's shape in FIG. 1 facilitates the passage of a tube 20 and stylet past the patient's tongue and away from the esophagus. Once the distal portion 12 is positioned at the vocal cords, its shape becomes detrimental to the passage of the tube 20. When the distal portion 12 reaches its predetermined temperature, it deflects posteriorly for a direct approach to the glottic opening, through the vocal cords, allowing the tube 20 to be passed over the improved intubation stylet in a more direct approach opposed to an anteriorly angled approach, which is a limitation of other stylets. The improved intubation stylet 10 does not Once the distal portion 12 is near the patient's vocal cords, the distal portion can be configured to deflect in a posterior direction to position the distal portion 12 directly at the vocal cords with minimal trauma. The heating of the distal portion 12 can be accomplished through various means, including, but not limited to: passively heating it using the body heat of a patient and/or applying heat through an external heater.

In the method, the distal portion 12 can further comprise nitinol. In the method, the distal portion 12 can be configured as a curved portion that at least partially reverses its direction of curvature. For example, the distal portion 12 can be manufactured with a curvature in a first direction 13 and configured to deflect in a second direction 14 when it reaches the predetermined temperature. The distal portion 12 can be configured as a straight portion that deflects into a curved portion. The distal portion can be configured as a straight portion that deflects in a posterior direction. The distal portion can be configured to be firm and easily moldable to a desirable shape prior to its deflection at the predetermined temperature. Due to manufacturing tolerances, the term “straight” includes variances of 10 degrees or curvature.

What has been described is an improved intubation stylet and methods of use that can reduce trauma when inserting breathing tubes in patients. In this disclosure, there are shown and described only exemplary embodiments of the invention, but, as aforementioned, it is to be understood that the invention is capable of use in various other combinations and environments and is capable of changes or modifications within the scope of the inventive concept as expressed herein.

From the foregoing disclosure and detailed description of certain preferred embodiments, it is also apparent that various modifications, additions and other alternative embodiments are possible without departing from the true scope and spirit. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the benefit to which they are fairly, legally, and equitably entitled.

What is claimed is:

1. An intubation stylet, which comprises:
a proximate portion configured to be firm and easily moldable;
a distal portion fixed to the proximate portion comprising a shape-memory alloy;
the distal portion having a thermal mass;
the distal portion having a curvature in a first direction and a shape memory in a second direction, where the second direction is opposite the first direction;
wherein at least a portion of the distal portion is configured to deflect from the first direction to the second direction at a predetermined temperature based in part on its thermal mass;
a tube slidably attached over the surface of the intubation stylet; and
wherein the intubation stylet is configured to also deflect the tube from the first direction to the second direction at the predetermined temperature.

2. The intubation stylet of claim 1, wherein the distal portion is configured to be firm and easily moldable prior to its deflection at the predetermined temperature.

3. The intubation stylet of claim 1, wherein the first direction is an anterior direction.

4. The intubation stylet of claim 3, wherein the second direction is a posterior direction.

5. The intubation stylet of claim 1, further comprising an external heat source fixed to an exterior surface of the intubation stylet and configured to heat the intubation stylet to the predetermined temperature.

6. A method of intubating a patient, the steps including:
providing an intubation stylet comprising:
a proximate portion configured to be easily moldable by hand;
a distal portion fixed to the proximate portion comprising a shape-memory alloy;
the distal portion having a thermal mass;
the distal portion having a curvature in a first direction and a shape memory in a second direction, where the second direction is opposite the first direction;
wherein at least a portion of the distal portion is configured to deflect from the first direction to the second direction at a predetermined temperature based in part on its thermal mass;
a tube slidably attached over a surface of the intubation stylet;
wherein the intubation stylet is configured to also deflect the tube from the first direction to the second direction at the predetermined temperature;
manipulating the proximate portion so that the distal portion points in an anterior direction;
deflecting the distal portion to a position that facilitates positioning the distal portion at a glottic opening of the patient by heating the distal portion to the predetermined temperature; and
sliding the tube over the intubation stylet and through the patient's vocal cords.

7. The method of claim 6, wherein the distal portion is configured to be firm and easily moldable prior to its deflection at the predetermined temperature.

8. The method of claim 6, wherein the first direction is an anterior direction.

9. The method of claim 8, wherein the second direction is a posterior direction.

10. The method of claim 6, wherein the intubation stylet further comprises an external heat source fixed to an exterior surface of the intubation stylet and configured to heat the intubation stylet to the predetermined temperature.
